(19) United States
(12) Patent Application Publication
(10) Pub. No.: US 2014/0005059 A1
(43) Pub. Date: Jan. 2, 2014

(54) BIOMARKERS FOR CANCER

(71) Applicants: Zhen Zhang, Dayton, MD (US); Jin Song, Baltimore, MD (US); Daniel Wan- Yui Chan, Clarksville, MD (US)

(72) Inventors: Zhen Zhang, Dayton, MD (US); Jin Song, Baltimore, MD (US); Daniel Wan- Yui Chan, Clarksville, MD (US)

(73) Assignee: The Johns Hopkins University, Baltimore, MD (US)

(21) Appl. No.: 12/530,456

(22) Filed: Sep. 8, 2009

Related U.S. Application Data
(63) Continuation of application No. PCT/US2008/056147, filed on Mar. 7, 2008.
(60) Provisional application No. 60/905,478, filed on Mar. 7, 2007.

(51) Int. Cl. G01N 33/574

(52) U.S. Cl. CPC G01N 33/57449
USPC 506/9; 435/7.92

(57) ABSTRACT

The instant invention provides methods and compositions for the diagnosis and treatment of cancer. The invention also provides method and compositions for determining if a subject is, or is at risk of becoming, chemoresistant.

### RELATED APPLICATIONS

This application claims the benefit of US Provisional Application No.: 60/905,478, entitled, “Biomarkers for Chemo-Resistance in Ovarian Cancer”, filed Mar. 7, 2007. The entire contents of the aforementioned provisional application are hereby incorporated by reference.

### GOVERNMENT SUPPORT

The following invention was supported at least in part by Department of Defense IDEA grant DAMD17-OC03-IDEA, NCI Early Detection Research Network (EDRN) grant CA115102-01, and NCI Grant 1P50 CA83639. Accordingly, the government has certain rights in the invention.

### BACKGROUND OF THE INVENTION

Ovarian cancer is the fifth leading cause of cancer death among U.S. women and has the highest mortality rate of all gynecologic cancers (1). Due to lack of effective screening tools and therapy, the mortality of ovarian cancer has not declined in the past two decades. Most cases of ovarian cancer, approximately 75%, are diagnosed at an advanced stage of the disease (1). While patients with early stage disease will have over a 74% chance of survival, those with advanced stage cancer will have overall survival rates of only 19-30% (1, 2). Administration of adjuvant chemotherapy consisting of a platinum compound (cisplatin or carboplatin) and a taxene remains the standard treatment for advanced stage cancer following an optimal primary debulking surgery (3). One of the most important clinical problems in the treatment of ovarian cancer is the intrinsic/acquired resistance to cisplatin-based chemotherapy. Although they are initially very responsive (80%) to cisplatin-based chemotherapy, 75% of patients easily develop cisplatin resistance and relapse within 2 years of primary therapy (4). The progression of cisplatin-resistant cancer confers poor prognosis and decreases overall survival of this disease.

Several mechanisms such as decreased drug accumulation, enhanced detoxification, drug sequestration, faster repair of cisplatin-DNA adducts and modulation of apoptotic pathways have been implicated in cisplatin resistance, but they are not sufficient to exhaustively explain this resistance emergence (5-9). Identification and characterization of more determinants of cisplatin resistance will advance our understanding of the varied mechanism that can contribute to this clinically relevant phenomenon, and lead to the development of new protein markers or to the establishment of new therapeutic strategies.

Accordingly, a need exists to better understand the molecular mechanism of chemoresistance in ovarian cancer subjects.

### SUMMARY OF THE INVENTION

The instant invention is based, at least in part, on the discovery by the inventors that a number of molecules are differentially expressed in cells that have become chemoresistant. Additionally, the inventors have found that the chance of recurrence of cancer is increased subjects that have become chemoresistant and who express altered levels of these biomarkers. The inventors have also found that subjects that produce autoantibodies to these biomarkers have cancer.

Accordingly, in one aspect, the instant invention provides methods of determining if a subject has become or is at risk of becoming chemoresistant, comprising obtaining a biological sample from the subject; and measuring the level of one or more proteins selected from the group consisting of translin-associated factor X (TRAX), nuclear domain 10 protein (NDP52), Na+/K+ ATPase b2, caspase-7/Mch3 and heat shock protein 60 (Hsp60), wherein an increased level of the protein is indicative that the subject is or will become chemoresistant.

In another aspect, the instant invention provides methods of determining if a subject has become or is at risk of becoming chemoresistant, comprising obtaining a biological sample from the subject; and measuring the level of one or more proteins selected from the group consisting of annexin A11, 5-hydroxytryptamine 2A receptor/Serotonin Receptor (5-HT2AR), Multi-PDZ-domain protein 1 (MUPP1), Monocyte chemotactic protein 1 (MCP-1), MUPP1, Cyclooxygenase-2 (COX-2/PGHS), miotogen-activated protein (MAP) Kinase 5 (MEK5), TRAF2 and NCK-interacting protein kinase (TNIK); and Diacylglycerol kinase theta (DGKq), wherein a decreased level of the protein is indicative that the subject is or will become chemoresistant.

In related embodiments, the one or more proteins is selected from the group consisting of annexin A11 and MUPP1. In a specific embodiment, the protein is annexin A11.

In related embodiments, the subjects are chemoresistant to a platinum based chemotherapeutic, e.g., Carboplatin, Cisplatin, Oxaliplatin, BBR3464, or Satraplatin. In a specific embodiment, the platinum based therapeutic is cisplatin.

In a related embodiment, the subject has a cell proliferative disorder, e.g., cancer. In exemplary embodiments, the cancer is pancreatic, kidney, stomach, colon, lung, bladder, prostate, uterine, breast or ovarian cancer. In a specific embodiment, the cancer is ovarian cancer.

In related embodiments, the increase or decrease of the level of the protein is relative to a control. In one embodiment, the control is a sample from a non-cancerous tissue.

In an exemplary embodiment, the invention provides a method of determining if a subject having ovarian cancer has become or is at risk of becoming chemoresistant, comprising obtaining a biological sample from the subject; and measuring the level of annexin A11, wherein a decreased level of annexin XI is indicative that the subject has or will become chemoresistant.

In one embodiment, the subject is chemoresistant to a platinum based chemotherapeutic, e.g., Carboplatin, Cisplatin, Oxaliplatin, BBR3464, and Satraplatin. In a preferred embodiment, the platinum based chemotherapeutic is cisplatin.

In one embodiment, the decrease in the level of the annexin A11 is relative to a control, e.g., a non-cancerous tissue.

In another aspect, the invention provides methods of determining if subject is likely to have a recurrence of cancer comprising obtaining a biological sample from the subject; and measuring the level of annexin A11 in the sample, wherein a decreased level of annexin XI is indicative that the subject will have a recurrence of cancer.

In another aspect, the invention also provides methods of treating a subject having cancer comprising administering to the subject a nucleic acid molecule encoding annexin A11, wherein the nucleic acid molecule is capable of producing annexin A11 in the cells of the subject. In one embodiment, the nucleic acid molecule is a nucleic acid vector, e.g., a viral vector.

In another embodiment, the nucleic acid molecule is administered with one or more chemotherapeutic molecules.

In another embodiment, the invention provides method of determining the prognosis of a subject having cancer comprising, obtaining a biological sample from the subject; and measuring the level of one or more proteins selected from the group consisting of translin-associated factor X (TRAX) nuclear domain 10 protein (NDP52); Na+/K+ ATPase b2, caspase-7/Mch3 and heat shock protein 60 (Hsp60), wherein an increased level of the protein is indicative of poor prognosis.

In another aspect, the invention provides method of determining the prognosis of a subject having cancer comprising obtaining a biological sample from the subject; and measuring the level of one or more proteins selected from the group consisting of annexin A11, 5-hydroxytryptamine 2A receptor/Serotonin Receptor (5-HT2AR), Multi-PDZ-domain protein 1 (MUPP1); Monocyte chemotactic protein 1 (MCP-1), MUPP1, Cyclooxygenase-2 (COX-2/PGHS), miotogen-activated protein (MAP) Kinase 5 (MEK5), TRAF2, NCK-interacting protein kinase (TNIK) and Diacylglycerol kinase theta (DGKq), wherein a decreased level of the protein is indicative of poor prognosis. In exemplary embodiments, the one or more proteins is selected from the group consisting of annexin A11 and MUPP1. In a specific embodiment, the protein is annexin A11.

In related embodiments, the subject is chemoresistant to a platinum based chemotherapeutic, e.g., Carboplatin, Cisplatin, Oxaliplatin, BBR3464, and Satraplatin. In a specific embodiment, the platinum based therapeutic is cisplatin.

In certain embodiments of the invention, the subjects have, or are suspected of having a cancer selected from of pancreatic, kidney, stomach, colon, lung, bladder, prostate, uterine, breast and ovarian cancer. In a specific embodiment, the cancer is ovarian cancer.

In certain embodiments, the increase or decrease of the level of the protein is relative to a control, e.g., a sample of a non-cancerous tissue.

In another aspect, the invention provides methods of determining the prognosis of a subject having ovarian cancer comprising, obtaining a biological sample from the subject; and measuring the level of annexin A11 in the sample, wherein a decreased level of the annexin A11 is indicative of poor prognosis.

In another aspect, the invention provides methods of diagnosing cancer comprising, obtaining a serum sample from a subject; and determining the levels of autoantibodies to annexin A11 in the sample, wherein elevated levels of autoantibodies in the sample is indicative that the subject has cancer. In one embodiment, the cancer is ovarian cancer.

In another aspect, the invention provides kits for the diagnosis of cancer comprising an antibody that specifically bind to annexin A11 and instructions for use.

In another aspect, the invention provides kits determining the prognosis of a subject having cancer comprising an antibody that specifically binds to annexin A11 and instructions for use.

In another aspect, the invention provides kits diagnosis of cancer comprising a reagent for the detection of autoantibodies to annexin A11 and instructions for use. In one embodiment, the reagent is a polypeptide. In another embodiment the polypeptide comprises the N-terminal domain of annexin A11, e.g., residues 1-175 of SEQ ID NO:2.

In another aspect, the invention provides method of detecting autoantibodies, which method is an immunoassay comprising contacting a sample to be tested for the presence of such autoantibodies with an immunoassay reagent and detecting the presence of complexes formed by specific binding of the immunoassay reagent to the cancer-associated autoantibodies present in the sample, wherein the immunoassay reagent comprises a polypeptide comprising the N-terminus of annexin A11 wherein said tumor marker protein exhibits selective reactivity with autoantibodies. In one exemplary embodiment, the N-terminus comprises residues 1-175 of annexin A11.

In one embodiment, the increased level of autoantibodies relative to a control is indicative of cancer, e.g., ovarian cancer.

### DESCRIPTION OF THE DRAWINGS

FIG. 1 demonstrates the use of the Clontech™ Ab Microarray 500 (Cat. No. 631790) to determine the relative protein abundance in cisplatin-sensitive and -resistant human ovarian cancer cells. The pseudo-color representation of the 16-bit Tiff files acquired from both slides depicts the relative protein abundances in 2008 vs 2008/C13*5.25. Red represents proteins with higher abundance in the Cy5 channel, while green represents proteins with higher abundance in the Cy3 channel. Yellow represents proteins with no apparent difference in the abundance between the two cell lines. Left: 2008-Cy5 vs 2008/C13*5.25-Cy3; right: 2008-Cy3 vs 2008/C13*5.25-Cy5. Red and green arrows point to antibodies detecting annexin A11.

FIG. 2A-B demonstrate validation of the antibody microarrays INR (internally normalized ratios) data by immunobloting. A. Immunobloting using the same monoclonal antibody as printed on the array (anti-annexin A11 mouse IgG1) showed a single band corresponding to annexin A11 protein mass in the pair of 2008 and 2008/C13*5.25 cell lines. B. The downregulation of expression of annexin A11 was confirmed in all three pairs of cells. DR represents drug-resistant cell line; DS represents drug-sensitive cell line.

FIG. 3A-J depict immunohistochemical staining of normal and malignant human tissues for annexin A11. A, normal pancreas; B, normal stomach; C, normal colon; D, normal breast; E, normal kidney; F, renal cell carcinoma; G, normal uterus; H, uterine endometrial adenocarcinoma; I, normal cerebrum; J, astrocytoma. A-E and G, positive; F and H-J, negative.

FIGS. 4A-F depict Immunohistochemical staining for annexin A11 in three representative pairs of surgical specimens derived from primary and matched recurrent ovarian cancers of the same patient. All three paired cases showed decreased expression of annexin A11 in recurrent tumors (B, D, and F) compared with their corresponding primary tumors (A, C, and E).

FIG. 5 depicts Kaplan-Meier survival curves of disease-free interval for patients with annexin A11-low- (solid line, ≦2) and high- (dashed line, >2) expressing tumors (p=0.03).

FIGS. 6A-B depict the nucleic acid and polypeptide sequence of annexin A11.

### DETAILED DESCRIPTION OF THE INVENTION

As used herein, the term “cancer” is used to mean a condition in which a cell in a patient's body undergoes abnormal, uncontrolled proliferation. Thus, “cancer” is a cell-proliferative disorder. Non-limiting examples of cancers include breast cancer, cervical cancer, prostate cancer, colon cancer, lung cancer, skin cancer, melanoma or any other type of cancer.

The terms “array” or “matrix” refer to an arrangement of addressable locations or “addresses” on a device. The locations can be arranged in two-dimensional arrays, three-dimensional arrays, or other matrix formats. The number of locations may range from several to at least hundreds of thousands. Most importantly, each location represents a totally independent reaction site. A “nucleic acid array” refers to an array containing nucleic acid probes, such as oligonucleotides or larger portions of genes.

“Biological activity” or “bioactivity” or “activity” or “biological function,” which are used interchangeably, herein mean an effector or antigenic function that is directly or indirectly performed by a polypeptide (whether in its native or denatured conformation), or by any subsequence thereof. Biological activities include binding to polypeptides, binding to other proteins or molecules, activity as a DNA binding protein, as a transcription regulator, ability to bind damaged DNA, etc. A bioactivity can be modulated by directly affecting the subject polypeptide. Alternatively, a bioactivity can be altered by modulating the level of the polypeptide, such as by modulating expression of the corresponding gene.

The term “sample” or “biological sample,” as used herein, refers to a sample obtained from an organism or from components (e.g., cells) of an organism. The sample may be of any biological tissue or fluid. The sample may be a sample which is derived from a patient. Such samples include, but are not limited to, sputum, blood, blood cells (e.g., white cells), tissue or biopsy samples (e.g., tumor biopsy), urine, peritoneal fluid, and pleural fluid, or cells therefrom. Biological samples may also include sections of tissues such as frozen sections taken for histological purposes. The terms refer to a sample of tissue or fluid isolated from an individual, preferably suspected of being afflicted with, or at risk of developing cancer. Such samples are primary isolates (in contrast to cultured cells) and may be collected by a non-invasive means, including, but not limited to, fine needle aspiration, needle biopsy, or another suitable means recognized in the art. Alternatively, the “sample” may be collected by an invasive method, including, but not limited to, surgical biopsy.

The term “biomarker” or “marker” encompasses a broad range of intra- and extra-cellular events as well as whole-organism physiological changes. Biomarkers may be represent essentially any aspect of cell function, for example, but not limited to, levels or rate of production of signaling molecules, transcription factors, metabolites, gene transcripts as well as post-translational modifications of proteins. Biomarkers may include whole genome analysis of transcript levels or whole proteome analysis of protein levels and/or modifications.

A biomarker may also refer to a gene or gene product which is up- or down-regulated in a compound-treated, diseased cell of a subject having the disease compared to an untreated diseased cell. That is, the gene or gene product is sufficiently specific to the treated cell that it may be used, optionally with other genes or gene products, to identify, predict, or detect efficacy of a small molecule. Thus, a biomarker is a gene or gene product that is characteristic of efficacy of a compound in a diseased cell or the response of that diseased cell to treatment by the compound. In specific embodiments, the biomarkers of the invention are those polypeptides that are differentially expressed in cancerous samples when compared to non-cancerous samples. In a specific embodiment, the biomarker of the invention is annexin A11.

A nucleotide sequence is “complementary” to another nucleotide sequence if each of the bases of the two sequences match, that is, are capable of forming Watson-Crick base pairs. The term “complementary strand” is used herein interchangeably with the term “complement.” The complement of a nucleic acid strand may be the complement of a coding strand or the complement of a non-coding strand.

The term “cancer” includes, but is not limited to, solid tumors, such as cancers of the breast, respiratory tract, brain, reproductive organs, digestive tract, urinary tract, eye, liver, skin, head and neck, thyroid, parathyroid, and their distant metastases. The term also includes lymphomas, sarcomas, and leukemias.

“Hybridization” refers to any process by which a strand of nucleic acid binds with a complementary strand through base pairing. For example, two single-stranded nucleic acids “hybridize” when they form a double-stranded duplex. The region of double-strandedness may include the fill-length of one or both of the single-stranded nucleic acids, or all of one single-stranded nucleic acid and a subsequence of the other single-stranded nucleic acid, or the region of double-strandedness may include a subsequence of each nucleic acid. Hybridization also includes the formation of duplexes which contain certain mismatches, provided that the two strands are still forming a double-stranded helix. “Stringent hybridization conditions” refers to hybridization conditions resulting in essentially specific hybridization.

The term “isolated,” as used herein, with respect to nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, culture medium when produced by recombinant DNA techniques, or chemical precursors or other chemicals when chemically synthesized. Moreover, an “isolated nucleic acid” may include nucleic acid fragments which are not naturally occurring as fragments and would not be found in the natural state. The term “isolated” is also used herein to refer to polypeptides which are substantially free of other cellular proteins and is meant to encompass both purified and recombinant polypeptides.

As used herein, the term “level of expression” refers to the measurable expression level of a given polypeptide or nucleic acid molecule. The level of expression of the polypeptide or nucleic acid is determined by methods well known in the art. The term “differentially expressed” or “differential expression” refers to an increase or decrease in the measurable expression level of a given polypeptide or nucleic acid. Absolute quantification of the level of expression of a polypeptide or nucleic acid may be accomplished by comparing the level to that of a control. The control can be an average amount of the molecule in a statistically significant number of samples, or can be compared to a the level of the molecule in a non-cancerous sample.

As used herein, the term “nucleic acid” refers to polynucleotides such as deoxyribonucleic acid (DNA) and, where appropriate, ribonucleic acid (RNA). The term should also be understood to include, as equivalents, analogs of either RNA or DNA made from nucleotide analogs and, as applicable to the embodiment being described, single-stranded (sense or antisense) and double-stranded polynucleotides. Chromosomes, cDNAs, mRNAs, rRNAs, and ESTs are representative examples of molecules that may be referred to as nucleic acids.

The term “oligonucleotide” as used herein refers to a nucleic acid molecule comprising, for example, from about 10 to about 1000 nucleotides. Oligonucleotides for use in the present invention are preferably from about 15 to about 150 nucleotides, more preferably from about 150 to about 1000 in length. The oligonucleotide may be a naturally occurring oligonucleotide or a synthetic oligonucleotide. Oligonucleotides may be prepared by the phosphoramidite method (Beaucage and Carruthers, Tetrahedron Lett. 22:1859-62, 1981), or by the triester method (Matteucci, et al., J. Am. Chem. Soc. 103:3185, 1981), or by other chemical methods known in the art.

The term “protein” is used interchangeably herein with the terms “peptide” and “polypeptide.”

As used herein, the term “cell-proliferative disorder” denotes malignant as well as non-malignant (or benign) disorders. This term further encompasses hyperplastic disorders. The cells comprising these proliferative disorders often appear morphologically and genotypically to differ from the surrounding normal tissue. As noted above, cell-proliferative disorders may be associated, for example, with chemoresistance. Expression of a biomarker of the invention, e.g., annexin A11 may be indicative of chemoresistance. The biomarkers of the invention, e.g., annexin A11, also provide information to the clinician as to the likelihood of recurrence of cancer. The finding that a subject has altered levels of a biomarker of the invention can influence the course of treatment that subject receives.

As used herein, the term “chemotherapeutic agents” refers to chemicals useful for the treatment of cell proliferative disorders. Chemotherapeutic agents may be categorized by their mechanism of action into, for example, the following groups: anti-metabolites/anti-cancer agents, such as pyrimidine analogs (5-fluorouracil, floxuridine, capecitabine, gemcitabine and cytarabine) and purine analogs, folate antagonists and related inhibitors (mercaptopurine, thioguanine, pentostatin and 2-chlorodeoxyadenosine (cladribine)); antiproliferative/antimitotic agents including natural products such as vinca alkaloids (vinblastine, vincristine, and vinorelbine), microtubule disruptors such as taxane (paclitaxel, docetaxel), vincristin, vinblastin, nocodazole, epothilones and navelbine, epidipodophyllotoxins (etoposide, teniposide), DNA damaging agents (actinomycin, amsacrine, anthracyclines, bleomycin, busulfan, camptothecin, carboplatin, chlorambucil, cisplatin, cyclophosphamide, cytoxan, dactinomycin, daunorubicin, doxorubicin, epirubicin, hexamethylmelamineoxaliplatin, iphosphamide, melphalan, merchlorehtamine, mitomycin, mitoxantrone, nitrosourea, plicamycin, procarbazine, taxol, taxotere, teniposide, triethylenethiophosphoramide and etoposide (VP16)); antibiotics such as dactinomycin (actinomycin D), daunorubicin, doxorubicin (adriamycin), idarubicin, anthracyclines, mitoxantrone, bleomycins, plicamycin (mithramycin) and mitomycin; enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nitrosoureas (carmustine (BCNU) and analogs, streptozocin), trazenes-dacarbazinine (DTIC); antiproliferative/antimitotic antimetabolites such as folic acid analogs (methotrexate); platinum coordination complexes (cisplatin, carboplatin), procarbazine, hydroxyurea, mitotane, aminoglutethimide; hormones, hormone analogs (estrogen, tamoxifen, goserelin, bicalutamide, nilutamide) and aromatase inhibitors (letrozole, anastrozole); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory agents; antisecretory agents (breveldin); immunosuppressives (cyclosporine, tacrolimus (FK-506), sirolimus (rapamycin), azathioprine, mycophenolate mofetil); anti-angiogenic compounds (TNP470, genistein) and growth factor inhibitors (vascular endothelial growth factor (VEGF) inhibitors, fibroblast growth factor (FGF) inhibitors); angiotensin receptor blocker; nitric oxide donors; anti-sense oligonucleotides; antibodies (trastuzumab, rituximab); cell cycle inhibitors and differentiation inducers (tretinoin); mTOR inhibitors, topoisomerase inhibitors (doxorubicin (adriamycin), amsacrine, camptothecin, daunorubicin, dactinomycin, eniposide, epirubicin, etoposide, idarubicin, irinotecan (CPT-11) and mitoxantrone, topotecan, irinotecan), corticosteroids (cortisone, dexamethasone, hydrocortisone, methylpednisolone, prednisone, and prenisolone); growth factor signal transduction kinase inhibitors; mitochondrial dysfunction inducers, toxins such as Cholera toxin, ricin, Pseudomonas exotoxin, Bordetella pertussis adenylate cyclase toxin, or diphtheria toxin, and caspase activators; and chromatin disruptors.

In preferred embodiments of the invention, the chemotherapeutic agent to which the subject becomes resistant to is a platinum based therapeutic, e.g., Carboplatin, Cisplatin, Oxaliplatin, BBR3464, Satraplatin. In a specific embodiment, the chemotherapeutic agent is cisplatin.

As used herein, the term, “chemoresistant” refers to subjects who fail to respond to the action of one or more chemotherapeutic agents. Most subjects are not chemoresistant at the beginning of treatment but may become so after a period of treatment. In specific embodiments, subjects that are chemoresistant are chemoresistant to platinum based therapeutics. In a particular embodiment, the subjects are chemoresistant to cisplatin.

Methods of Detecting the Biomarkers of the Invention

The biomarkers of the invention can be nucleic acid or polypeptide biomarkers. In a preferred embodiment, the biomarkers are polypeptides. The instant invention is based on the finding that certain molecules are differentially expressed in cells that have become, or are becoming, chemoresistant. In order to determine if a cell is chemoresistant, of at risk of becoming chemoresistant, the instant invention provides methods for determining the level of the identified biomarkers in a biological sample. Specifically, the invention provides methods and compositions for determining the amount of a protein or nucleic acid biomarker of the invention in a biological sample.

In clinical applications, human tissue samples may be screened for the presence and/or absence of biomarkers identified herein. Such samples could consist of needle biopsy cores, surgical resection samples, lymph node tissue, or serum. For example, these methods include obtaining a biopsy, which is optionally fractionated by cryostat sectioning to enrich tumor cells to about 80% of the total cell population. In certain embodiments, nucleic acids extracted from these samples may be amplified using techniques well known in the art. The levels of selected markers detected could be compared with statistically valid normal tissue samples.

In one embodiment, the diagnostic method comprises determining whether a subject has an abnormal nucleic acid and/or protein level of the biomarkers, such as by Northern blot analysis, reverse transcription-polymerase chain reaction (RT-PCR), in situ hybridization, immunoprecipitation, Western blot hybridization, or immunohistochemistry. According to the method, cells may be obtained from a subject and the levels of the biomarkers, protein, or nucleic acid level, are determined and compared to the level of these markers in a healthy subject. An abnormal level of the biomarker polypeptide or nucleic acid levels is indicative of chemoresistance.

Accordingly, in one aspect, the invention provides probes and primers that are specific to the unique nucleic acid markers disclosed herein. Accordingly, the nucleic acid probes comprise a nucleotide sequence at least 10 nucleotides in length, preferably at least 15 nucleotides, more preferably, 25 nucleotides, and most preferably at least 40 nucleotides, and up to all or nearly all of the coding sequence which is complementary to a portion of the coding sequence of a marker nucleic acid sequence.

The invention further provides a method of determining whether a sample obtained from a subject possesses an abnormal amount of a biomarker of the invention comprising (a) obtaining a sample from the subject, (b) quantitatively determining the amount of the biomarker in the sample, and (c) comparing the amount of the marker polypeptide so determined with a known standard or to a control, thereby determining whether the sample obtained from the subject possesses an abnormal amount of the marker polypeptide. Such marker polypeptides may be detected by immunohistochemical assays, dot-blot assays, ELISA, and the like.

Immunoassays are commonly used to quantitate the levels of proteins in cell samples, and many other immunoassay techniques are known in the art. The invention is not limited to a particular assay procedure, and therefore, is intended to include both homogeneous and heterogeneous procedures. Exemplary immunoassays which may be conducted according to the invention include fluorescence polarization immunoassay (FPIA), fluorescence immunoassay (FIA), enzyme immunoassay (EIA), nephelometric inhibition immunoassay (NIA), enzyme-linked immunosorbent assay (ELISA), and radioimmunoassay (RIA). An indicator moiety, or label group, may be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

In another embodiment, the level of the encoded product, or alternatively the level of the polypeptide, in a biological fluid (e.g., blood or urine) of a patient may be determined as a way of monitoring the level of expression of the marker nucleic acid sequence in cells of that patient. Such a method would include the steps of obtaining a sample of a biological fluid from the patient, contacting the sample (or proteins from the sample) with an antibody specific for an encoded marker polypeptide, and determining the amount of immune complex formation by the antibody, with the amount of immune complex formation being indicative of the level of the marker encoded product in the sample. This determination is particularly instructive when compared to the amount of immune complex formation by the same antibody in a control sample taken from a normal individual or in one or more samples previously or subsequently obtained from the same person.

The term “antibody” as used herein includes antibodies that react with a biomarker of the invention, e.g., annexin A11 or with one or more peptide fragments of a biomarker of the invention. The term “antibodies” is also intended to include parts thereof such as Fab, Fv fragments as well as antibodies that react with the overlapping regions of one or more of the peptide fragments of the invention and recombinantly produced fragments and fusion products of antibody fragments (including multivalent and/or multi-specific). The term “antibodies” is also intended to include antibodies to receptors specific for one or more of the peptide fragments of the invention. Antibodies can be fragmented using conventional techniques and the fragments screened for utility in the same manner as described above. Antibodies may be used either for screening for diagnostic purposes or in order to identify additional peptide fragments, mimetics, variants and inhibitors of the invention. Exemplary commercially available antibodies to the biomarkers of the invention are set forth in Table 2.

The term “autoantibody” refers to an antibody obtained from an individual or animal and which is reactive to a normal cellular antigen(s) or a self-antigen from the same individual or animal. Specific autoantibodies of the invention are those that react with annexin A11.

Conventional methods can be used to prepare the antibodies. For example, by using a peptide of the invention, polyclonal antisera or monoclonal antibodies can be made using standard methods. This invention also contemplates chimeric antibody molecules, made by methods known to those skilled in the art.

The antibodies may be labeled with a detectable marker including various enzymes, fluorescent materials, luminescent materials and radioactive materials as is known to those skilled in the art.

Antibodies reactive against naturally occurring biomarkers of the invention and fragments thereof (e.g., enzyme conjugates or labeled derivatives) may be used to detect a biomarker of the invention, including the peptide sequence in various samples, such as tissue or body fluid samples. For example, they may be used in any known immunoassays and immunological methods that rely on the binding interaction between an antigenic determinant of a protein of the invention and the antibodies. Examples of such assays are radioimmunoassays, Western immunoblotting, enzyme immunoassays (e.g. ELISA), immunofluorescence, immunoprecipitation, latex agglutination, and immunohistochemical tests. Thus, the antibodies may be used to identify or quantify the amount of a biomarker of the invention in a sample and thus may be used as a diagnostic indicator of chemoresistance.

A sample may be tested for the presence or absence of a biomarker of the invention by contacting the sample with an antibody specific for an epitope, e.g., an epitope of Annexin A11, which antibody is capable of being detected after it becomes bound to a biomarker of the invention in the sample, and assaying for antibody bound to a biomarker of the invention in the sample.

In the method of the immunoassay, a predetermined amount of a biological sample or concentrated sample is mixed with antibody or labelled antibody. The amount of antibody used in the method is dependent upon the labelling agent chosen. The amount of a biomarker of the invention bound to antibody or labelled antibody may then be detected by methods known to those skilled in the art. The sample or antibody may be insolubilized, for example, the sample or antibody can be reacted using known methods with a suitable carrier. Examples of suitable carriers are Sepharose or agarose beads. When an insolubilized sample or antibody is used, a biomarker of the invention bound to antibody or unreacted antibody is isolated by washing. For example, when the sample is blotted onto a nitrocellulose membrane, the antibody bound to a biomarker of the invention is separated from the unreacted antibody by washing with a buffer, for example, phosphate buffered saline (PBS) with bovine serum albumin (BSA).

When labeled antibody is used, the presence of a biomarker of the invention can be determined by measuring the amount of labeled antibody bound in the sample. The appropriate method of measuring the labeled material is dependent upon the labeling agent.

The methods of the invention may be performed on any related tissue or body fluid sample. In one embodiment, the sample is preferably a ovarian tissue sample. Alternatively, the methods of the invention can be performed on a body fluid sample selected from the group consisting of blood, plasma, serum, fecal matter, urine, semen, seminal fluid or plasma.

Preferred according to the present invention is annexin A11, including fragments thereof, and conservatively substituted variants thereof.

Polyclonal and monoclonal antibodies of the invention are immunoreactive with a biomarker of the invention or immunogenic fragments of a biomarker of the invention.

The term “antibody” also includes any synthetic or genetically engineered protein that is functionally capable of binding an epitopic determinant of a biomarker of the invention. It also refers to a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment.

An “antibody fragment” is a portion of an antibody such as F(ab′)₂, F(ab)₂, Fab′, Fab, Fv, scFv (single chain Fv) and the like. Regardless of structure, an antibody fragment binds with to same antigen that is recognized by the intact antibody.

The term “antibody fragment” also includes any synthetic or genetically engineered protein that acts like an antibody by binding to a specific biomarker antigen to form a complex. For example, antibody fragments include isolated fragments consisting of the variable regions, such as the “Fv” fragments consisting of the variable regions of the heavy and light chains, recombinant single chain polypeptide molecules in which light and heavy variable regions are connected by a peptide linker (“scFv proteins”), and minimal recognition units consisting of the amino acid residues that mimic the hypervariable region. The Fv fragments may be constructed in different ways as to yield multivalent and/or multispecific binding forms. In the former case of multivalent, they react with more than one binding site against the specific epitope, whereas with multispecific forms, more than one epitope (either of the antigen or even against the specific antigen and a different antigen) is bound.

A “chimeric antibody” is a recombinant protein that contains the variable domains of both the heavy and light antibody chains, including the complementarity determining regions (CDRs) of an antibody derived from one species, preferably a rodent antibody, while the constant domains of the antibody molecule are derived from those of a human antibody. For veterinary applications, the constant domains of the chimeric antibody may be derived from that of other species, such as a cat or dog.

A “humanized antibody” is a recombinant protein in which the CDRs from an antibody from one species, e.g., a rodent antibody, is transferred from the heavy and light variable chains of the rodent antibody into human heavy and light variable domains. The constant domains of the antibody molecule are derived from those of a human antibody.

A “human antibody” is an antibody obtained from transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13 (1994), Lonberg et al., Nature 368:856 (1994), and Taylor et al., Int. Immun. 6:579 (1994). A fully human antibody also can be constructed by genetic or chromosomal transfection methods, as well as phage display technology, all of which are known in the art. See for example, McCafferty et al., Nature 348:552-553 (1990) for the production of human antibodies and fragments thereof in vitro, from immunoglobulin variable domain gene repertoires from unimmunized donors. In this technique, antibody variable domain genes are cloned in-frame into either a major or minor coat protein gene of a filamentous bacteriophage, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. In this way, the phage mimics some of the properties of the B cell. Phage display can be performed in a variety of formats, for their review, see e.g., Johnson and Chiswell, Current Opinion in Structural Biol. 3:5564-571 (1993).

For purposes of the invention, an antibody or nucleic acid probe specific for an EPCA may be used to detect the presence of the a biomarker of the invention (in the case of an antibody probe) or polynucleotide (in the case of the nucleic acid probe) in biological fluids or tissues. Oligonucleotide primers based on any coding sequence region of a biomarker of the invention are useful for amplifying DNA or RNA, for example by PCR. The term “amplification” as used herein, relates to the production of additional copies of a nucleic acid sequence. Amplification is generally carried out using polymerase chain reaction (PCR) technologies that are well known in the art. (See, e.g., Dieffenbach, C. W. and G. S. Dveksler (1995), PCR Primer, a Laboratory Manual, Cold Spring Harbor Press, Plainview, N.Y., pp. 1-5). Any specimen containing a detectable amount of EPCA antigen can be used. A preferred sample of this invention is tissue taken from the prostate. Alternatively, biological fluids which may contain cells of the prostate may be used.

Methods that directly compare the qualitative and quantitative protein content of tumor and normal cells are known in the art. These methods include immunoassays, one-dimensional and two-dimensional gel electrophoresis characterization, western blotting, matrix assisted laser desorption/time of flight (MALDI/TOF) mass spectrometry, liquid chromatography quadruple ion trap electrospray (LCQ-MS) and surface enhanced laser desorption ionization/time of flight (SELDI/TOF) mass spectrometry. These methods coupled with the laser capture microdissection method of Liotta et al. (WO 00/49410) can determine the protein characteristics of a biological sample. These methods can be used to deter mine the level of a biomarker of the invention in a sample, i.e., the level in a biological sample v. a control sample.

The present invention contemplates using the above-mentioned methods to compare the protein of the present invention in normal and test samples. Biomarkers of the invention can be used either alone or in combination with a ligand, such as a monoclonal antibody. For example, SELDI can be used in combination with a time-of-flight mass spectrometer (TOF) to provide a means to rapidly analyze a biomarker of the invention or peptide fragments thereof retained on a chip (Hutchens and Yip, Rapid Commun. Mass Spectrom. 7:576-580, 1993). SELDI/TOF can be applied to ligand-protein interaction analysis by covalently binding the target protein on the chip and using mass spectroscopy to analyze the small molecules that bind to the target protein (Worrall et al. Anal Biochem. 70:750-756, 1998).

The immunological processes of a human subject may produce auto-antibodies directed to the protein of the present invention (annexin A11), as a result of a cell proliferative disorder, e.g., cancer. These antibodies, directed to a self-derived protein, would be an autoantibodies by definition. As such, autoantibodies can be measured in body fluids or tissues by immunological in vitro diagnostic methods wherein the biomarker of the invention protein or antigenic fragments thereof can be used as target substrates. The detection of, for example, annexin A11 auto-antibodies may correlate with the pathological state of cancer and, therefore, would be useful for diagnostic purposes.

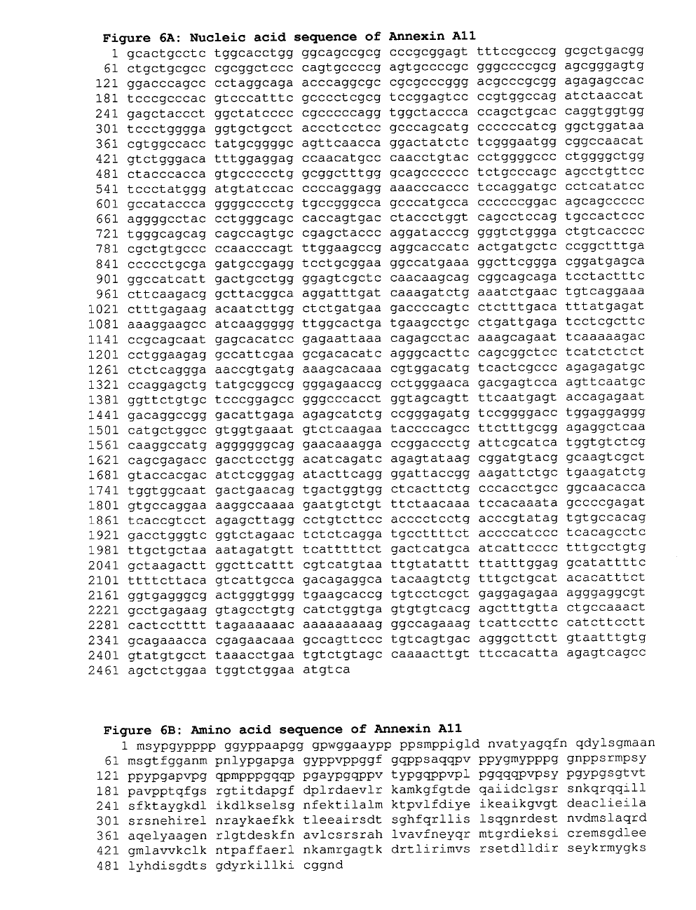

Auto-antibodies reactive with for example, annexin A11, can be measured by a variety of immunoassay methods. For a review of immunological and immunoassay procedures in general, see Basic and Clinical Immunology, 7th Edition, D. Stites and A. Terr (ed.), 1991; “Practice and Theory of Enzyme Immunoassays,” P. Tijssen, Laboratory Techniques in Biochemistry and Molecular Biology, Elsevier Science Publishers, B. V., Amsterdam (1985); and Harlow and Lane, Antibodies, A Laboratory Manual. The entire contents of these references are incorporated herein by reference. Specifically, annexin A11 autoantibodies can be measured using the assay described in the examples using a polypeptide comprising the N-terminal domain of annexin A11. In a specific embodiment, the protein comprise residues 1-175 of annexin A11 (genbank accession number NP_{—}001148). The nucleic acid and amino acid sequences of annexin A11 are set forth in FIGS. 6A-B.

The invention also provides methods of determining expression levels of various genes in the biological samples as described above and comparing the expression levels with the expression level in a control sample.

The method for determining the expression levels of genes is not particularly limited, and any of techniques for confirming alterations of the gene expressions mentioned above can be suitably used.

In an exemplary method, mRNA is prepared from a biological sample, and then reverse transcription is carried out with the resulting mRNA as a template. During this process, labeled cDNA can be obtained by using, for instance, any suitable labeled primers or labeled nucleotides.

### Methods of Treatment

In on embodiment, the invention provides methods and compositions for treating a cell-proliferative disorder, e.g., ovarian cancer. In one embodiment, the instant invention provides methods for treating a subject having ovarian cancer by administering to a subject an effective amount of a compound that inhibits the activity of autoantibodies to annexin A11.

The instant invention provides detailed teachings that decreased levels of certain polypeptides result in the subject becoming chemoresistant, having a poor prognosis, a decreased length of survival, and/or a increased risk of recurrence. Accordingly, methods that increase the level of the polypeptide to near wild-type levels would be useful to treat these subjects. In a particular embodiment, subjects who are chemoresistant to one or more chemotherapeutics are administered a polynucleotide that results in increased expression of annexin A11. In a particular embodiment, the subject is chemoresistant to cisplatin.

The therapeutic polynucleotides and polypeptides of the present invention can be delivered using gene delivery vehicles. The gene delivery vehicle can be of viral or non-viral origin (see generally, Jolly, Cancer Gene Therapy (1994) 1:51; Kimura, Human Gene Therapy (1994) 5:845; Connelly, Human Gene Therapy (1995) 1:185; and Kaplitt, Nature Genetics (1994) 6:148). Expression of such coding sequences can be induced using endogenous mammalian or heterologous promoters. Expression of the coding sequence can be either constitutive or regulated.

Viral-based vectors for delivery of a desired polynucleotide and expression in a desired cell are well known in the art. Exemplary viral-based vehicles include, but are not limited to, recombinant retroviruses (see, e.g., WO 90/07936; WO 94/03622; WO 93/25698; WO 93/25234; U.S. Pat. No. 5,219,740; WO 93/11230; WO 93/10218; U.S. Pat. No. 4,777,127; GB Patent No. 2,200,651; EP 0 345 242; and WO 91/02805), alphavirus-based vectors (e.g., Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532)), and adeno-associated virus (AAV) vectors (see, e.g., WO 94/12649, WO 93/03769; WO 93/19191; WO 94/28938; WO 95/11984 and WO 95/00655). Administration of DNA linked to killed adenovirus as described in Curiel, Hum. Gene Ther. (1992) 3:147 can also be employed.

Non-viral delivery vehicles and methods can also be employed, including, but not limited to, polycationic condensed DNA linked or unlinked to killed adenovirus alone (see, e.g., Curiel, Hum. Gene Ther. (1992) 3:147); ligand-linked DNA (see, e.g., Wu, J. Biol. Chem. (1989) 264:16985); eukaryotic cell delivery vehicles cells (see, e.g., U.S. Pat. No. 5,814,482; WO 95/07994; WO 96/17072; WO 95/30763; and WO 97/42338) and nucleic charge neutralization or fusion with cell membranes. Naked DNA can also be employed. Exemplary naked DNA introduction methods are described in WO 90/11092 and U.S. Pat. No. 5,580,859. Liposomes that can act as gene delivery vehicles are described in U.S. Pat. No. 5,422,120; WO 95/13796; WO 94/23697; WO 91/14445; and EP 0524968. Additional approaches are described in Philip, Mol. Cell. Biol. (1994) 14:2411, and in Woffendin, Proc. Natl. Acad. Sci. (1994) 91:1581.

Further non-viral delivery suitable for use includes mechanical delivery systems such as the approach described in Woffendin et al., Proc. Natl. Acad. Sci. USA (1994) 91(24): 11581. Moreover, the coding sequence and the product of expression of such can be delivered through deposition of photopolymerized hydrogel materials or use of ionizing radiation (see, e.g., U.S. Pat. No. 5,206,152 and WO 92/11033). Other conventional methods for gene delivery that can be used for delivery of the coding sequence include, for example, use of hand-held gene transfer particle gun (see, e.g., U.S. Pat. No. 5,149,655); use of ionizing radiation for activating transferred gene (see, e.g., U.S. Pat. No. 5,206,152 and WO 92/11033).

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Annexin A11 is Associated with Cisplatin Resistance and Related to Tumor Recurrence in Ovarian Cancer Patients

Ovarian cancer has been generally treated with cisplatin-based chemotherapy and often recurs due to acquired cisplatin resistance. The precise nature of cisplatin resistance remains unclear. The goal of this study was to identify proteins that are differentially expressed in multiple pairs of cisplatin-sensitive and -resistant human ovarian cancer cell lines. The protein expression was further investigated in ovarian cancer tissues to address its clinical significance.

Experimental Design:

Antibody microarrays were used to identify proteins consistently differentially expressed across 3 pairs of cisplatin-sensitive and -resistant ovarian cancer cell lines. The alteration of the protein was validated by immunoblotting. The protein expression was further evaluated by immunohistochemical staining using tissue microarrays containing various human normal and malignant tissues, and 164 surgical specimens derived from primary and recurrent ovarian cancer patients who underwent optimal primary debulking surgery followed by a standard chemotherapeutic regimen.

Summary of Results:

Annexin A11 was downregulated in all three cisplatin-resistant cell lines as compared to their parent cell lines. Annexin A11 was expressed in the majority of human normal organs including pancreas, kidney, stomach, colon, lung, bladder, prostate, uterus, placenta, breast, and ovary. The decreased expression of annexin A11 was observed in some of the most common human malignancies relative to their cognate normal tissues. The expression level of annexin A11 in first recurrent ovarian cancers was much lower than that in primary ovarian cancers (p=0.0004). Among 20 pairs of surgical specimens derived from primary and matched recurrent ovarian cancers of the same patient, 14 paired cases showed reduced expression of annexin A11 in recurrent lesions, whereas only 2 paired cases showed a reverse alteration. Among 40 advanced-stage serous carcinomas, patients with low annexin A11-expressing tumors exhibited earlier recurrence than those with high annexin A11-expressing tumors (p=0.03). Annexin A11 immunoreactivity negatively correlated with in vitro cisplatin resistance in ovarian cancers (p=0.01).

### Materials and Methods

Cell Lines and Culture.

A total of six human ovarian cancer cell lines (A2780, A2780cis, 2008, 2008/C13*5.25, HEY, and HEY C2) were used in this study. Sublines stably resistant to cisplatin (A2780cis, 2008/C13*5.25, and HEY C2) had been prepared from each parental cell line by repeated in vitro exposure to cisplatin as previously described (12). A2780 and A2780cis cells were purchased from ECACC. 2008, 2008/C13*5.25, HEY, and HEY C2 cells were kindly provided by Dr. S. B. Howell (Department of Medicine, University of California-San Diego, La Jolla, Calif.). All cell lines were maintained in drug-free RPMI-1640 medium (Gibco/Invitrogen, Carlsbad, Calif.) supplemented with 10% (v/v) heat-inactivated fetal bovine serum (Hyclone, Logan, Utah) and 1% penicillin-streptomycin (Gibco/Invitrogen, Carlsbad, Calif.) at 37° C. in a humidified atmosphere containing 5% CO₂.

Cell Cytotoxicity Assay.

The sensitivities of the cells to cisplatin were determined by a Cell Counting Kit-8 (Dojindo Molecular Technologies, Gaithersburg, Md.). Briefly, 100 μl of cell suspension (10⁴ cells/well) were dispensed in 96-well microplates and incubated overnight at 37° C. in a humidified incubator containing 5% CO₂. Then the cells were treated with various concentrations of cisplatin diluted in 100 μl of conditioned medium (the final concentrations of cisplatin were 0, 1.56, 3.13, 6.25, 12.5, 25, 50, 100, and 200 μg/ml). After incubation for 48 hours, 10 μl of Cell Counting Kit-8 solution was added to each well, the plates were further incubated for 4 hours at 37° C. The absorbance at 450 nm was measured with a microplate reader (EL 312e Biokinetics reader, Biotek Instruments, Winooski, Vt.). Three independent experiments were performed for each pair of cell lines.

Antibody Microarray. Clontech™ Antibody (Ab) Microarray 500 (Cat. No. 631790; Clontech Laboratories, Mountain View, Calif.) was used to determine the protein expression profiles in three pairs of cell lines. The array is composed of more than 500 distinct monoclonal antibodies printed at high density on a glass slide in duplicate, which cover five major functional categories based on gene ontology: apoptosis, cancer, cell cycle, protein kinases, and neurobiology. A list of the antibodies on the array can be found from the Clontech™ website.

Measuring protein abundances with Ab Microarrays consists of five main steps: extracting protein, labeling protein with Cy5 and Cy3 dyes, removing unbound dye, incubating labeled protein with the arrays, and scanning arrays to measure bound antigen. Briefly, protein extraction and labeling were performed using Clontech™ Ab Microarray Buffer Kit (Cat. No. 631791) following the manufacturer's protocol. About 50-150 mg of cell pellets was thawed and homogenized in non-denaturing Extraction/Labeling buffer especially formulated and without protease inhibitors. The protein concentration of each extract was measured with the BCA Protein Assay Reagent Kit (Pierce Biotechnology, Rockford, Ill.). Each sample (e.g. sample A, drug-sensitive cell line; sample B, drug-resistant cell line) was then diluted to 1.1 mg protein/ml by adding the appropriate volume of Extraction/Labeling buffer. To control for differences in labeling efficiency, sample A and B were each split into two equal portions. Each portion was then labeled with either Cy5 or Cy3 (Amersham Biosciences, Pittsburgh, Pa.) to produce four samples: A-Cy5, A-Cy3, B-Cy5, and B-Cy3 (i.e. 450 μl protein extract+25 μl dye+25 μl Extraction/Labeling buffer). After protein labeling, unbound dye was removed by gel exclusion chromatography (gel filtration) using a disposable PD-10 desalting column (Amersham Biosciences, Pittsburgh, Pa.). Labeled proteins were collected by elution with 2.0 ml of 1× desalting buffer. Protein concentrations of labeled samples were determined using the BCA method. The average number of dye molecules covalently coupled to each protein (Dye-to-protein ratio) was estimated following the manufacturer's protocol, which should be in the range of 2-4. These four samples (50 mg/each) were combined to produce two mixtures (100 μg/each) of Cy5- and Cy3-labeled proteins: A-Cy5 is combined with B-Cy3 (Mix1), while A-Cy3 is combined with B-Cy5 (Mix2). The two mixtures (20 μg/each) were then hybridized with the two identical Ab Microarray slides in separate incubation chambers at room temperature for 30 minutes: slide 1 is hybridized with Mix1 and slide 2 is hybridized with Mix2. All steps were performed as described by the manufacturer. The slides were washed, dried and scanned under the optimized PMT settings using a GenePix 4000B scanner (Axon Instruments, Foster City, Calif.).

Grid alignment and initial quantification of array images were performed using GenePix Pro 3.0 software. Signal intensities for each coordinate on the array were determined by background-subtracted median intensities from Cy5 and Cy3 channels. Array 1 measures A-Cy5/B-Cy3 (Ratio 1) and array 2 measures B-Cy5/A-Cy3 (Ratio 2). The derived Cy5/Cy3 ratios were imported into the Ab Microarray Analysis Workbook (Microsoft Excel 97/98; Clontech Laboratories, Mountain View, Calif.). An average internally normalized ratio (INR, the square root of average ratio 1/ratio 2) is calculated, which represents the abundance of an antigen in sample A relative to that of sample B. Using the Ab Microarray Workbook, average values (e.g. average ratio 1/ratio 2 and average INR) are considered invalid if they are based on duplicates that differ by more than 30%. INR values are considered invalid if they are based on Cy5/Cy3 ratios in which one or more of the antigen signals are less than twice the background signal. The average INR for the experiment was then obtained by averaging the INR values of all antigens. The average INR for the experiment was multiplied by 1.3 to obtain the upper threshold value and 0.77 to obtain the lower threshold value for that experiment (11, 13). Proteins with INR values outside this interval were considered differentially expressed in the pair of cells. An INR > the upper threshold value indicates that an antigen is more abundant in cisplatin-sensitive cell line than cisplatin-resistant cell line. Conversely, an INR < the lower threshold value indicates that an antigen is less abundant in cisplatin-sensitive cell line than cisplatin-resistant cell line. The protein consistently differentially expressed across multiple pairs of cells was subjected to further analysis.

Immunoblot Analysis.

All three pairs of cells were used to validate the above antibody microarrays results. Protein extraction and concentration measurement were performed as described above. The denatured samples (40 or 80 μg of protein) of cisplatin-sensitive and -resistant cell lines were electrophoresed on 4-15% gradient SDS-PAGE gels (Bio-Rad, Hercules, Calif.), electroblotted on nitrocellulose membranes (Bio-Rad, Hercules, Calif.), and probed with the same monoclonal antibody as printed on the arrays (anti-annexin A11 mouse IgG1, clone 16, 1:10,000; BD Biosciences, San Jose, Calif.). The bound antibodies were visualized with horse-radish peroxidase-conjugated secondary antibodies and enhanced chemiluminescence (Amersham Biosciences, Pittsburgh, Pa.). Actin in the corresponding cell lysates was used as an additional control to show equal loading. Three independent experiments were performed for each pair of cell lines.

Human Subjects and Tissue Specimens.

In accordance with the human subject research guidelines of institutional review board, formalin-fixed, paraffin-embedded tissues were obtained from the Department of Pathology at Johns Hopkins Medical Institutions (JHMI). These included 164 ovarian carcinoma tissues, which are 93 primary tumors, 57 first recurrent tumors, and 14 second or third recurrent tumors (Table 1). All patients underwent primary debulking surgery followed by platinum/paclitaxel-based combined chemotherapy. Among 57 first recurrent patients, 20 pairs of surgical specimens were derived from primary and matched recurrent lesions of the same patient. The TMAs were constructed as described previously (14). Briefly, tissue cores (1.5 mm in diameter) were taken from three spatially separate areas in a single donor block from each case using a tissue microarrayer (Beecher Instruments, Silver Spring, Md.). Cores were precisely arrayed into a recipient paraffin block at defined coordinates to form an array of 11×9 cores format. The selection of areas for cores and the confirmation of the presence of tumor on TMAs were made by a pathologist (I-M.S.) based on review of the corresponding H&E slides from donor and recipient paraffin blocks. In addition, tissue arrays containing normal pancreas, kidney, stomach, colon, lung, bladder, prostate, uterus, placenta, breast, ovary, liver, cerebrum, testis, thyroid, thymus, and lymph node and arrays containing tumor tissue from cancers of the pancreas, kidney, stomach, colon, lung, bladder, prostate, uterus, breast, ovary, liver, cerebrum, testis, thyroid, lymph node, fibrous tissues, skin, and head and neck were obtained from US Biomax, Inc. (Rockville, Md.).

TABLE I
Clinicopathologic characteristics of the study cohort.

| Variables | No. of patients | % |
|---|---|---|
| Total | 164 | |
| Primary | 93 | 56.7 |
| 1^{st} recurrent | 57 | 34.8 |
| 2^{nd} or 3^{rd} recurrent | 14 | 8.5 |
| Age (y) | | |
| Mean | 55 | |
| Range | 23-88 | |
| FIGO^{a} Stage | | |
| I | 12 | 7.3 |
| II | 7 | 4.3 |
| III | 128 | 78 |
| IV | 17 | 10.4 |
| Grade | | |
| GB^{b} | 3 | 1.8 |
| 1 | 15 | 9.1 |
| 2 | 20 | 12.2 |
| 3 | 96 | 58.5 |
| NA^{c} | 30 | 18.3 |
| Histotype | | |
| Endometrioid | 3 | 1.8 |
| Serous | 142 | 86.6 |
| Others^{d} | 19 | 11.6 |

^{a}FIGO, International Federation of Gynecologists and Obstetricians.
^{b}GB, borderline cancer.
^{c}NA, relevant clinical information not available.
^{d}Others, mixed epithelial carcinomas or not otherwise specified.

Immunohistochemistry.

The EnVision+System-HRP (DAB) (DakoCytomation, Carpinteria, Calif.) was used in this study. Four-micrometer histological sections were deparaffinized and rehydrated in graded ethanols. Antigen retrieval was performed by placing slides in 10 mM sodium citrate buffer (pH 6.0) inside a preheated steamer (95-100° C.) for 25 min. Endogenous peroxidase activity was blocked by 0.03% hydrogen peroxide for 10 min. Sections were then incubated with the same monoclonal antibody as printed on the arrays (anti-annexin A11 mouse IgG1, clone 16, 1:200; BD Biosciences, San Jose, Calif.) at room temperature for 40 min. After incubation, the specimens were washed with 1× TBST buffer (DakoCytomation, Carpinteria, Calif.) and incubated with peroxidase labelled polymer at room temperature for 40 min. The samples were then washed with 1×TBST buffer and incubated with freshly prepared DAB+substrate-chromogen buffer at room temperature for 3 min. After gently rinsing with dH₂O, slides were counterstained with hematoxylin (DakoCytomation, Carpinteria, Calif.) and mounted with permanent mounting media (DakoCytomation, Carpinteria, Calif.). For negative controls, the primary antibody was omitted and replaced by pre-immune mouse IgG1 serum (DakoCytomation, Carpinteria, Calif.), respectively.

Evaluation of IHC Staining Results.

The IHC staining of the protein in the panel of ovarian carcinoma tissues were scored semi-quantitatively from 0 to 3+ as follows: negative (0), weak (1+), moderate (2+), and strong (3+) expression. Briefly, both the percentage of stained cells (0, ≦10%; 1, 11-25%; 2, 26-50%; 3, 51-75%; 4, 76-90%; 5, ≧91%) and the intensity of the staining (0, none; 1, weak; 2, moderate; 3, strong) were assessed in every component on every slide as described previously (15, 16). A final score was obtained by combining the percentage of stained cells with the intensity of the staining as follow: samples with an intensity level of 0 or 1 in ≦10% of cells were designated negative; samples with an intensity level of 1 in >10% of cells or with intensity levels of 2-3 and the added scores of 2-3 were designated weak; samples with intensity levels of 2-3 and the added scores of 4-6 or 7-8 were designated moderate or strong, accordingly. Each core on TMAs was scored individually and then the final score for each case was determined by combining the results of replicate cores for that case. In addition, various normal tissues were considered positive for the protein expression if any staining was detectable. Various malignant tissues were considered positive for the protein expression if and staining was detectable in malignant cells (17).

Extreme Drug Resistance Assay.

In vitro drug responses of tumors were assessed by the extreme drug resistance (EDR) assay (Oncotech, Tustin, Calif.) (18). Briefly, fresh tumor specimens immediately removed at the time of surgery were placed into the RPMI (Life Technologies, Gaithersburg, Md.) and shipped to Oncotech for analysis. The tumor tissue was minced and enzymatically digested to disaggregate the tumor cell, which were then plated in soft agar. Tumor cells were then exposed to antineoplastic agents (cisplatin, carboplatin, paclitaxel, and taxotere) at a concentration of a maximum tolerated dose for 5 days in vitro. Tritiated thymidine was introduced during the last 2 days of culture as a measure of cell proliferation, and the drug-treated cells were compared with the untreated control. Assay results were divided into three categories: extreme, intermediate, and low drug resistance as described previously (18).

Statistical Analyses.

On the basis of available clinicopathologic information including age, stage, grade, histological type, treatment history, recurrence status, survivorship, and EDR status, Fisher's exact test was performed to compare the protein differential expression levels in ovarian carcinomas. Survival curves were established using the Kaplan-Meier method, and their differences were analyzed using the log-rank test. Overall survival time was defined as the number of months between diagnosis and death. Disease-free interval was defined as the number of months between primary surgery and first tumor recurrence. Differences with P<0.05 were considered statistically significant. All of the statistical analyses were performed using Statistica 6.1 (Statsoft).

### Results

Sensitivity to Cisplatin.

Three pairs of human ovarian cancer cell lines (A2780 vs A2780cis, 2008 vs 2008/C13*5.25, and HEY vs HEY C2) were used in this study. Each pair consisted of a cisplatin-sensitive parental cell line and a subline that had been selected for stable acquired cisplatin resistance by repeated in vitro exposure to cisplatin. Cell cytotoxicity assay were performed to determine the sensitivity of the three pair of cisplatin-sensitive and -resistant cell lines to the cytotoxic effect of cisplatin. Dose-response curves were plotted on a semi-log scale as the percentage of the control cell number, which was obtained from the sample without drug exposure. Resistance to cisplatin in A2780cis, 2008/C13*5.25, and HEY C2 cell lines was confirmed (P<0.05, data not shown).

Determination of Protein Abundances in Cisplatin-Sensitive and -Resistant Human Ovarian Cancer Cells.

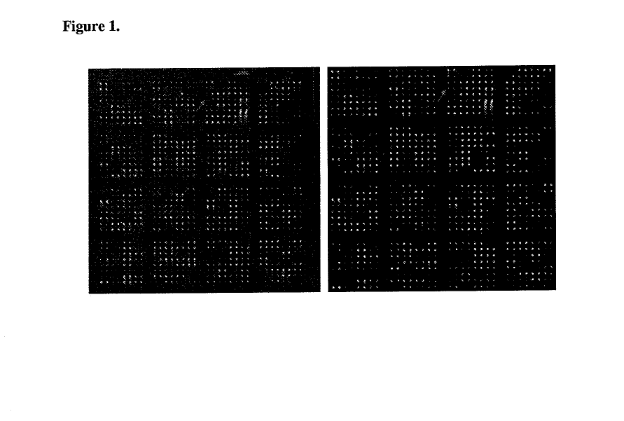

Using antibody microarrays, expression levels of 500 proteins representing a broad range of functional classes in three pairs of cells were simultaneously measured (FIG. 1). A panel of proteins differentially expressed in three pairs of cells and associated with cisplatin resistance were identified (Table 2). The respective threshold values of INR for three pairs of cells were as follow: 0.98-1.66 (2008 vs 2008/C13*5.25), 0.73-1.23 (HEY vs HEY C2), and 0.99-1.67 (A2780 vs A2780cis). Proteins with INR values outside the interval were considered differentially expressed in the pair of cells. Proteins with higher abundance in cisplatin-resistant cell lines were translin-associated factor X (TRAX) and nuclear domain 10 protein (NDP52) for 2008/C13*5.25; Na+/K+ ATPase b2 and caspase-7/Mch3 for Hey C2; heat shock protein 60 (Hsp60) for A2780cis. Proteins with higher abundance in cisplatin-sensitive cell lines were annexin A11, 5-hydroxytryptamine 2A receptor/Serotonin Receptor (5-HT2AR), and Multi-PDZ-domain protein 1 (MUPP1) for 2008; Monocyte chemotactic protein 1 (MCP-1), MUPP1, Cyclooxygenase-2 (COX-2/PGHS), annexin A11, miotogen-activated protein (MAP) Kinase 5 (MEK5), and TRAF2 and NCK-interacting protein kinase (TNIK) for Hey; Diacylglycerol kinase theta (DGKq) for A2780 (Table 2). Among these, annexin A11 and MUPP1 were consistently downregulated in two cisplatin-resistant cell lines (2008/C13*5.25 and HEY C2) compared to their parent cell lines (2008 and HEY).

TABLE 2
Determination of protein abundances in cisplatin-sensitive and -resistant
human ovarian cancer cells (A2780 vs A2780cis, 2008 vs
2008/C13*5.25, and HEY vs HEY C2) using antibody microarrays.

| | SwissProt | | |
|---|---|---|---|
| Antigen | Accession # | Clontech Ab ID | INR |
| Higher abundance in 2008/C13*5.25 | | | |
| TRAX | Q99598 | AB_001262 | 0.75 |
| NDP52 | Q13137 | AB_000266 | 0.94 |
| Higher abundance in 2008 | | | |
| Annexin A11 | P50995 | AB_001152 | 1.68 |
| 5-HT2AR | P28223 | AB_000901 | 1.79 |
| MUPP1 | O75970 | AB_001200 | 1.80 |
| Higher abundance in HEY C2 | | | |
| Na+/K+ ATPase b2 | P14415 | AB_000264 | 0.69 |
| Caspase-7/Mch3 | P55210 | AB_001001 | 0.70 |
| Higher abundance in HEY | | | |
| MCP-1 | P13500 | AB_001269 | 1.25 |
| MUPP1 | O75970 | AB_001200 | 1.26 |
| COX-2/PGHS | P35354 | AB_000538 | 1.28 |
| Annexin A11 | P50995 | AB_001152 | 1.32 |
| MEK5 | Q92961 | AB_000226 | 1.43 |
| Higher abundance in | Q9UKE5 | AB_001313 | 1.59 |
| A2780cis | | | |
| Hsp60 | P10809 | AB_001285 | 0.98 |
| Higher abundance in A2780 | | | |
| DGKq | P52824 | AB_000603 | 1.77 |

INR, internally normalized ratios.

Validation of Microarray Data by Immunoblotting.

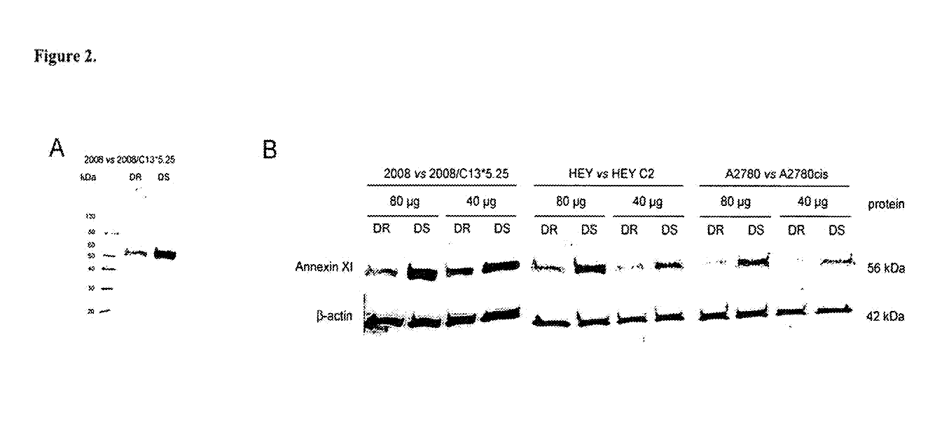

Annexin A11 is a member of the annexin superfamily of Ca²⁺ and phospholipid-binding, membrane-associated proteins implicated in Ca²⁺-signal transduction processes associated with cell growth and differentiation (19-22). Previously, annexin IV has been found less expressed in the cisplatin-resistant cell lines (IGROV1-R10 and IGROV1/CP) in comparison with the sensitive parental cell line (IGROV1, another ovarian cancer cell line) (23, 24). Therefore, we subsequently validated the alteration of annexin A11 in cisplatin-resistant ovarian cancer cell lines by immunobloting. Immunobloting using the same monoclonal antibody as printed on the array showed a single band corresponding to annexin A11 protein mass (FIG. 2A). Comparing with their parent cell lines, annexin A11 was found decreased not only in above two cell lines of 2008/C13*5.25 and HEY C2, but also in the third cisplatin-resistant cell line of A2780cis (FIG. 2B). The amount of downregulation of annexin A11 in three pairs of cells was estimated to be 3- to 8-fold using Quantity One 4.5 software (Bio-Rad, Hercules, Calif.).

Expression of Annexin A11 in Human Normal and Malignant Tissues.

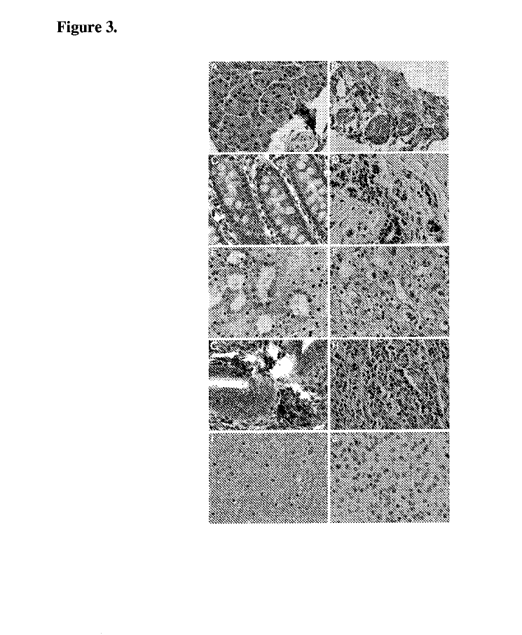

To investigate the tissue expression pattern of annexin A11, arrays containing various human normal tissues were stained with the same monoclonal antibody as used above. The experimental results showed that annexin A11 was expressed in majority of human normal organs including pancreas (11/12), kidney (10/11), stomach (8/9), colon (10/12), lung (9/13), bladder (4/5), prostate (9/10), uterus (5/5), placenta (2/4), breast (3/6), and ovary (2/4), but was not detected in liver (0/6), cerebrum (0/6), testis (0/4), thyroid (0/5), thymus (0/4), and lymph node (0/9). Examples of both positive (pancreas, stomach, colon, breast, kidney, and uterus) and negative (cerebrum) staining for annexin A11 in normal tissues are presented in FIG. 3 (A-E, G and I).

In addition, arrays containing various human malignancies were examined to determine whether expression of annexin A11 was altered in cancer tissues compared with the normal tissue from which they arose. The decreased expression of annexin A11 was observed in some of the most common human malignancies as compared with their cognate normal tissues. These tumor types included pancreas (12/18), kidney (6/20), stomach (13/18), colon (10/20), lung (7/15), bladder (11/18), prostate (2/10), uterus (9/19), breast (7/24), and ovary (11/31) (FIG. 3E-H). No staining was detectable in cancer tissues of cerebrum (0/18), testis (0/18), fibrous tissues (0/20), and head and neck (0/17) (FIG. 3J). Expression was only detected in a low fraction of cancer tissues of liver (3/19), thyroid (4/19), lymph node (4/18), and skin (2/14).

Decreased Expression of Annexin A11 Correlates with In Vitro Cisplatin Resistance and Tumor Recurrence in Ovarian Cancer Patients.

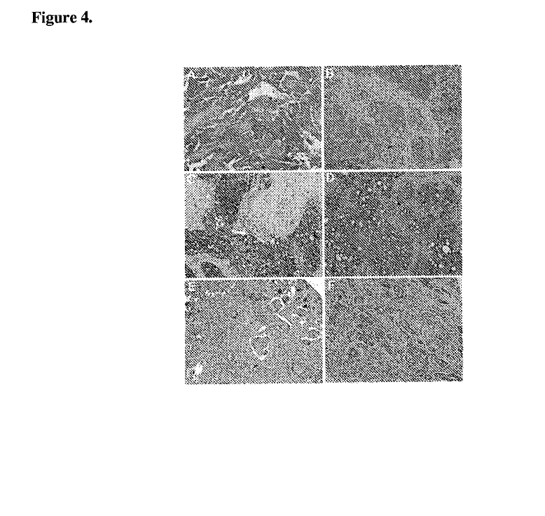

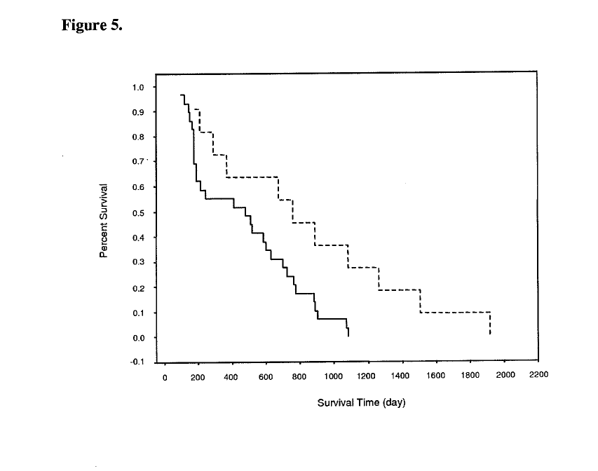

Ovarian cancer has been generally treated with cisplatin-based chemotherapy and often recurs due to acquired cisplatin resistance. To clinically confirm the involvement of annexin A11 in acquired cisplatin resistance, the expression of annexin A11 in 164 ovarian carcinoma tissues using immunohistochemical staining was evaluated. As shown in Table 3, the expression level of annexin A11 in first recurrent ovarian cancers (57 cases) was much lower than that in primary ovarian cancers (93 cases) (p=0.0004). Among 20 pairs of surgical specimens derived from primary and matched recurrent ovarian cancers of the same patients, 14 paired cases showed reduced expression of annexin A11 in recurrent lesions, whereas only 2 paired cases showed a reverse alteration (FIG. 4). A statistically significant decrease in annexin A11 immunointensity (0 and 1+) was found in recurrent tumors as compared with the primary tumors from the same patients (p=0.02). Annexin A11 immunostaining was observed in both cytoplasm and nucleus of cancerous epithelial cells contrasting with the adjacent immunonegative stroma cells (FIG. 4). No significant annexin A11 immunoreactivity was detected in negative controls that were analyzed with primary antibody omitted and pre-immune serum, respectively (data not shown). Among 40 patients with advanced-stage serous carcinomas who underwent primary debulking surgery followed by a standard chemotherapeutic regimen, patients with low annexin A11-expressing tumors (29 cases, ≦2) exhibited earlier recurrence than those with high annexin A11-expressing tumors (11 cases, >2) (p=0.03) (FIG. 5). The median disease-free interval with annexin A11 immunointensity of ≦2 was 473 days, whereas when the intensity was >2, the interval was 755 days. Among 105 tumors for which the EDR results were available for analysis, annexin A11 immunoreactivity negatively correlated with in vitro cisplatin resistance (p=0.01) (Table 4). The statistical significance in the association of annexin A11 immunoreactivity and in vitro cisplatin resistance even existed if only high grade (2 or 3), advanced stage (III or IV) serous carcinomas (67 tumors) were analyzed (p=0.02) (Table 4). However, expression of annexin A11 did not correlate with the status of in vitro drug resistance for carboplatin (p. 0.15), paclitaxel (p=0.57), and taxotere (p=0.78). There was no significant association between annexin A11 staining either in primary tumors or in recurrent tumors with either overall survival or any of the other clinical parameters including stage, grade, or histological subtype.

TABLE 3
Decreased annexin A11 immunoreactivity correlates with tumor
recurrence in ovarian cancer patients (P = 0.0004).

| Tumor | Negative | Weak | Moderate | Strong | Total |
|---|---|---|---|---|---|
| Primary | 3 (3.23%) | 37 (39.78%) | 30 (32.26%) | 23 (24.73%) | 93 |
| Recurrent | 9 (15.79%) | 34 (59.65%) | 8 (14.04%) | 6 (10.53%) | 57 |
| Total | 12 | 71 | 38 | 29 | 150 |

### Discussion

Cisplatin is the most common therapeutic agent used for chemotherapy in ovarian cancer. However, acquisition of cisplatin resistance during chemotherapy is mainly related to cancer mortality and remains a major clinical challenge. Cisplatin is a cytotoxic compound which causes apoptosis via DNA damage by formation of interstrand or intrastrand adducts. The response to cisplatin is a complex and multifactorial process that leads to the activation of several pathways organized in a large network and transmitting pro- or anti-apoptotic signals (24). The major mechanisms of resistance that have been identified so far involve reduced drug uptake, increased drug efflux, increased repair of platinum-DNA adducts, increased tolerance of DNA damage, and increased levels of intracellular thiols such as glutathione and metallothionein (5-9, 25).

In conclusion, this study showed that annexin A11 is associated with acquired cisplatin resistance in ovarian cancer. The decreased expression of annexin A11 is characteristic for cisplatin-resistant cancer cells and may directly contribute to tumor recurrence and progression. Accordingly, Annexin A 11 expression is a predictive marker of chemoresistance for platinum-based chemotherapy and is useful for identifying ovarian cancer patients who are likely to develop early recurrence.

### Example 2

### Epigenetic Silencing of Annexin A11 Confers Chemoresistance to Ovarian Cancer Cells Through Modulating Cell Proliferation

In order to confirm the results obtained in Example 1, RNA knockdown experiments were done to test the effects of decreased annexin A11 expression.

Up to 80% of patients diagnosed at advanced stages of ovarian cancer die within five years. Many of them, after undergoing debulking surgery, initially respond to chemotherapy yet later relapse with recurrent tumors that are refractory to the original treatment, eventually succumbing to the disease.

As demonstrated in Example 1, annexin A11 is associated with cisplatin resistance and related to tumor recurrence in ovarian cancer patients. More specifically, through a proteomic profiling, annexin A11 was found down-regulated in drug-resistant cells across three pairs of drug-sensitive and -resistant ovarian cancer cell lines.

To further decipher the molecular mechanism underlying annexin A11's effect on drug resistance in ovarian cancer, in the present study, an RNAi knockdown was developed of annexin A11 ovarian cancer cell models. The effect and duration of silencing annexin A11 expression on two human ovarian cancer cell lines, 2008 and HEY, were confirmed using immunoblot. Cell cytotoxicity, cell proliferation and colony formation assays were performed on these cells. Using these engineered cell models, it was demonstrated that epigenetic silencing of annexin A11 conferred chemoresistance to ovarian cancer cells (P<0.01), which confirms the results observed in Example 1.

It was further observed that the suppression of annexin A11 expression upregulated cyclin D1 and p21^{Cip/WAF1} expressions, and reduced cell proliferation (P<0.05) and colony formation ability (P<0.01) of cancer cells. These results demonstrate that the observed association between annexin A11 and drug resistance may be mediated through alterations in cell cycle/proliferation. It has been demonstrated both in vitro and clinically, tumor cells that undergo a growth arrest or have a lower proliferation activity may be protected from apoptosis and may therefore be ultimately resistant to the chemotherapeutic agents.

This study demonstrated that epigenetic silencing of annexin A11 confers chemoresistance to ovarian cancer cells.

### Example 3

### Prevalence and Characteristics of Autoantibodies to Annexin A11 in Different Types of Human Cancer

The following experiment was performed to determine if autoantibody production to annexin A11 was associated with cancer.

Annexin A11 is a member of the annexin superfamily of Ca2+ and phospholipids-binding, membrane-associated proteins implicated in Ca2+-signal transduction processes associated with cell growth and differentiation.

Previously, annexin A11 was identified as an autoantigen in 4.1-10.1% of patients with various systemic autoimmune diseases. The majority of these anti-annexin A11 autoantibodies belong to the IgG class, consistent with an antigen driven mechanism of autoantibody production, in contrast to that the autoantibodies to other annexins are primarily of the IgM isotype. Anti-annexin A11 autoantibodies do not cross-react with other annexin members, corroborating that they recognize the unique N-terminal domain of annexin A11. It is believed that autoantibodies can be viewed as reporters from the immune system revealing the identity of antigens, which might be playing roles in the pathophysiology of the disease process.

How annexin A11 participates in the pathogenesis of human cancers and whether a similar mechanism in autoimmune diseases might be involved in human immune responses in cancer remains to be established.

In this experiment, a novel enzyme-linked immunosorbent assay (ELISA) was developed to investigate the occurrence and features of autoantibodies against annexin A11 in sera from patients with different types of human cancer and diabetes as well as from healthy controls. Briefly, the recombinant protein of GST fused to the N-terminal domain (1-175 residues) of human annexin A11 (GST-Anx11N) was expressed in E. coli BL21 cells and purified by affinity chromatography using Glutathione-Sepharose 4B. The fusion protein was then used as antigen in ELISA and western blot for the detection of autoantibodies to annexin A11. A total of 246 serum specimens archived at the Johns Hopkins Hospital were analyzed, which includes sera from 77 healthy women; 72 patients with stage III/IV ovarian cancer (40 primary and 32 recurrent tumors); 18 patients with breast cancer; 19 patients with colon cancer; and groups of 20 patients each with pancreatic cancer, prostate cancer, or diabetes.

The overall titer of autoantibodies to annexin A11 in ovarian cancer patients (or primary tumors only) was found much higher than that in healthy controls (P<0.05). At the cut-off value (mean OD+2SD of healthy controls) designating positive reaction, autoantibodies to annexin A11 were detected in 12.5% (5/40) of primary ovarian cancer patients with a significant difference from 2.6% (2/77) of the healthy controls (P<0.05), but only in 6.25% (2/32) of recurrent tumors. ROC analysis demonstrated the potential diagnostic value of autoantibodies to annexin A11 in primary ovarian cancer patients with an AUC of 0.62 (0.52-0.73). Autoantibodies to annexin A11 were also detected in 5.26% (1/19) of colon cancer and 10% (2/20) of diabetes patients but without significant difference from the healthy controls.

This study demonstrated that anti-annexin A11 autoantibodies frequently occur in primary ovarian cancer patients in contrast to healthy controls, and maybe involved in the pathogenesis of ovarian cancer.

### REFERENCES

1. Jemal, A., Siegel, R., Ward, E., Murray, T., Xu, J., Smigal, C., and Thun, M. J. Cancer statistics, 2006. CA Cancer J Clin, 56: 106-130, 2006.
2. Holschneider, C. H., and Berek, J. S. Ovarian cancer: epidemiology, biology, and prognostic factors. Semin Surg Oncol, 19: 3-10, 2000.
3. Bristow, R. E., Tomacruz, R. S., Armstrong, D. K., Trimble, E. L., and Montz, F. J. Survival effect of maximal cytoreductive surgery for advanced ovarian carcinoma during the platinum era: a meta-analysis. J Clin Oncol, 20: 1248-1259, 2002.
4. Tummala, M. K., and McGuire, W. P. Recurrent ovarian cancer. Clin Adv Hematol Oncol, 3: 723-736, 2005.
5. Siddik, Z. H. Cisplatin: mode of cytotoxic action and molecular basis of resistance. Oncogene, 22: 7265-7279, 2003.
6. Boulikas, T., and Vougiouka, M. Cisplatin and platinum drugs at the molecular level. (Review). Oncol Rep, 10: 1663-1682, 2003.
7. Fink, D., Aebi, S., and Howell, S. B. The role of DNA mismatch repair in drug resistance. Clin Cancer Res, 4: 1-6, 1998.
8. Johnson, S. W., Laub, P. B., Beesley, J. S., Ozols, R. F., and Hamilton, T. C. Increased platinum-DNA damage tolerance is associated with cisplatin resistance and cross-resistance to various chemotherapeutic agents in unrelated human ovarian cancer cell lines. Cancer Res, 57: 850-856, 1997.
9. Perez, R. P., Hamilton, T. C., Ozols, R. F., and Young, R. C. Mechanisms and modulation of resistance to chemotherapy in ovarian cancer. Cancer, 71: 1571-1580, 1993.
10. Li, X., Wang, H., Touma, E., Rousseau, E., Quigg, R. J., and Ryaby, J. T. Genetic network and pathway analysis of differentially expressed proteins during critical cellular events in fracture repair. J Cell Biochem, 100: 527-543, 2007.
11. Andersson, O., Kozlowski, M., Garachtchenko, T., Nikoloff, C., Lew, N., Litman, D. J., and Chaga, G. Determination of relative protein abundance by internally normalized ratio algorithm with antibody arrays. J Proteome Res, 4: 758-767, 2005.
12. Mishima, M., Samimi, G., Kondo, A., Lin, X., and Howell, S. B. The cellular pharmacology of oxaliplatin resistance. Eur J Cancer, 38: 1405-1412, 2002.
13. Sreekumar, A., Nyati, M. K., Varambally, S., Barrette, T. R., Ghosh, D., Lawrence, T. S., and Chinnaiyan, A. M. Profiling of cancer cells using protein microarrays: discovery of novel radiation-regulated proteins. Cancer Res, 61: 7585-7593, 2001.
14. Yen, M. J., Hsu, C. Y., Mao, T. L., Wu, T. C., Roden, R., Wang, T. L., and Shih Ie, M. Diffuse mesothelin expression correlates with prolonged patient survival in ovarian serous carcinoma. Clin Cancer Res, 12: 827-831, 2006.
15. Bouras, T., Southey, M. C., and Venter, D. J. Overexpression of the steroid receptor coactivator AIB1 in breast cancer correlates with the absence of estrogen and progesterone receptors and positivity for p53 and HER2/neu. Cancer Res, 61: 903-907, 2001.
16. Armes, J. E., Trute, L., White, D., Southey, M. C., Hammet, F., Tesoriero, A., Hutchins, A. M., Dite, G. S., McCredie, M. R., Giles, G. G., Hopper, J. L., and Venter, D. J. Distinct molecular pathogeneses of early-onset breast cancers in BRCA1 and BRCA2 mutation carriers: a population-based study. Cancer Res, 59: 2011-2017, 1999.
17. Samimi, G., Varki, N. M., Wilczynski, S., Safaei, R., Alberts, D. S., and Howell, S. B. Increase in expression of the copper transporter ATP7A during platinum drug-based treatment is associated with poor survival in ovarian cancer patients. Clin Cancer Res, 9: 5853-5859, 2003.
18. Cloven, N. G., Kyshtoobayeva, A., Burger, R. A., Yu, I. R., and Fruehauf, J. P. In vitro chemoresistance and biomarker profiles are unique for histologic subtypes of epithelial ovarian cancer. Gynecol Oncol, 92: 160-166, 2004.
19. Monastyrskaya, K., Babiychuk, E. B., Hostettler, A., Rescher, U., and Draeger, A. Annexins as intracellular calcium sensors. Cell Calcium, 2006.
20. Chetcuti, A., Margan, S. H., Russell, P., Mann, S., Millar, D. S., Clark, S. J., Rogers, J., Handelsman, D. J., and Dong, Q. Loss of annexin II heavy and light chains in prostate cancer and its precursors. Cancer Res, 61: 6331-6334, 2001.
21. Brichory, F. M., Misek, D. E., Yim, A. M., Krause, M. C., Giordano, T. J., Beer, D. G., and Hanash, S. M. An immune response manifested by the common occurrence of annexins I and II autoantibodies and high circulating levels of IL-6 in lung cancer. Proc Natl Acad Sci USA, 98: 9824-9829, 2001.
22. Yeatman, T. J., Updyke, T. V., Kaetzel, M. A., Dedman, J. R., and Nicolson, G. L. Expression of annexins on the surfaces of non-metastatic and metastatic human and rodent tumor cells. Clin Exp Metastasis, 11: 37-44, 1993.
23. Stewart, J. J., White, J. T., Yan, X., Collins, S., Drescher, C. W., Urban, N. D., Hood, L., and Lin, B. Proteins associated with Cisplatin resistance in ovarian cancer cells identified by quantitative proteomic technology and integrated with mRNA expression levels. Mol Cell Proteomics, 5: 433-443, 2006.
24. Le Moguen, K., Lincet, H., Deslandes, E., Hubert-Roux, M., Lange, C., Poulain, L., Gauduchon, P., and Baudin, B. Comparative proteomic analysis of cisplatin sensitive IGROV1 ovarian carcinoma cell line and its resistant counterpart IGROV1-R10. Proteomics, 6: 5183-5192, 2006.
25. Cheng, T. C., Manorek, G., Samimi, G., Lin, X., Berry, C. C., and Howell, S. B. Identification of genes whose expression is associated with cisplatin resistance in human ovarian carcinoma cells. Cancer Chemother Pharmacol, 58: 384-395, 2006.

### INCORPORATION BY REFERENCE

The contents of all references, patents, pending patent applications and published patents, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method of determining if a subject has become or is at risk of becoming chemoresistant, comprising:
obtaining a biological sample from the subject; and
measuring the level of one or more proteins selected from the group consisting of translin-associated factor X (TRAX), nuclear domain 10 protein (NDP52); Na+/K+ ATPase b2, caspase-7/Mch3 and heat shock protein 60 (Hsp60),
wherein an increased level of the protein is indicative that the subject is or will become chemoresistant.

2. A method of determining if a subject has become or is at risk of becoming chemoresistant, comprising:
obtaining a biological sample from the subject; and
measuring the level of one or more proteins selected from the group consisting of annexin A11, 5-hydroxytryptamine 2A receptor/Serotonin Receptor (5-HT2AR), Multi-PDZ-domain protein 1 (MUPP1); Monocyte chemotactic protein 1 (MCP-1), MUPP1, Cyclooxygenase-2 (COX-2/PGHS), miotogen-activated protein (MAP) Kinase 5 (MEK5), TRAF2 and NCK-interacting protein kinase (TNIK); and Diacylglycerol kinase theta (DGKq);
wherein a decreased level of the protein is indicative that the subject is or will become chemoresistant.

3. The method of claim 2, wherein the one or more proteins is selected from the group consisting of annexin A11 and MUPP1.

4. The method of claim 3, wherein the protein is annexin A11.

5. The method of claim 1, wherein the subject is chemoresistant to a platinum based chemotherapeutic.

6. The method of claim 5, wherein the platinum based therapeutic is selected from the group consisting of: Carboplatin, Cisplatin, Oxaliplatin, BBR3464, and Satraplatin.

7. (canceled)

8. The method of claim 1, wherein the subject has a cell proliferative disorder.

9. The method of claim 8, wherein the cell proliferative disorder is cancer.

10-13. (canceled)

14. A method of determining if a subject having ovarian cancer has become or is at risk of becoming chemoresistant, comprising:
obtaining a biological sample from the subject; and
measuring the level of annexin A11;
wherein a decreased level of anneixin XI is indicative that the subject is or will become chemoresistant.

15. The method of claim 14, wherein the subject is chemoresistant to a platinum based chemotherapeutic.

16-19. (canceled)

20. A method of determining if subject is likely to have a recurrence of cancer comprising:
obtaining a biological sample from the subject; and
measuring the level of the annexin A11 polypeptide in the sample;
wherein a decreased level of annexin XI is indicative that the subject will have a recurrence of cancer.

21. A method of treating a subject having cancer comprising:
administering to the subject a nucleic acid molecule encoding annexin A11, wherein the nucleic acid molecule is capable of producing annexin A11 in the cells of the subject.

22. The method of claim 21, wherein the nucleic acid molecule is a nucleic acid vector.

23-24. (canceled)

25. A method of determining the prognosis of a subject having cancer comprising:
obtaining a biological sample from the subject; and
measuring the level of one or more proteins selected from the group consisting of translin-associated factor X (TRAX) nuclear domain 10 protein (NDP52); Na+/K+ ATPase b2, caspase-7/Mch3 and heat shock protein 60 (Hsp60),
wherein an increased level of the protein is indicative of poor prognosis.

26. A method of determining the prognosis of a subject having cancer comprising:
obtaining a biological sample from the subject; and
measuring the level of one or more proteins selected from the group consisting of annexin A11, 5-hydroxytryptamine 2A receptor/Serotonin Receptor (5-HT2AR), Multi-PDZ-domain protein 1 (MUPP1); Monocyte chemotactic protein 1 (MCP-1), MUPP1, Cyclooxygenase-2 (COX-2/PGHS), miotogen-activated protein (MAP) Kinase 5 (MEK5), TRAF2, NCK-interacting protein kinase (TNIK) and Diacylglycerol kinase theta (DGKq);
wherein a decreased level of the protein is indicative of poor prognosis.

27. The method of claim 26, wherein the one or more proteins is selected from the group consisting of annexin A11 and MUPP1.

28. The method of claim 3, wherein the protein is annexin A11.

29-36. (canceled)

37. A method of diagnosing cancer comprising:
obtaining a serum sample from a subject; and
determining the levels of autoantibodies to annexin A11 in the sample;
wherein elevated levels of autoantibodies in the sample is indicative that the subject has cancer.

38. (canceled)

39. A kit for the diagnosis of cancer comprising an antibody that specifically bind to annexin A11 and instructions for use.

40-43. (canceled)

44. A method of detecting autoantibodies, which method is an immunoassay comprising contacting a sample to be tested for the presence of such autoantibodies with an immunoassay reagent and detecting the presence of complexes formed by specific binding of the immunoassay reagent to the cancer-associated autoantibodies present in the sample, wherein the immunoassay reagent comprises a polypeptide comprising the N-terminus of annexin A11 wherein said tumor marker protein exhibits selective reactivity with autoantibodies.

45-47. (canceled)


SEQUENCE LISTING (2 sequences)

SEQ ID NO: 1 - DNA, 2486 bp, Homo sapiens
gcactgcctctggcacctggggcagccgcgcccgcggagttttccgcccggcgctgacggctgctgcgcccgcggctccccagtgccccgagtgccccgcgggccccgcgagcgggagtgggacccagcccctaggcagaacccaggcgccgcgcccgggacgcccgcggagagagccactcccgcccacgtcccatttcgcccctcgcgtccggagtccccgtggccagatctaaccatgagctaccctggctatcccccgcccccaggtggctacccaccagctgcaccaggtggtggtccctggggaggtgctgcctaccctcctccgcccagcatgccccccatcgggctggataacgtggccacctatgcggggcagttcaaccaggactatctctcgggaatggcggccaacatgtctgggacatttggaggagccaacatgcccaacctgtaccctggggcccctggggctggctacccaccagtgccccctggcggctttgggcagcccccctctgcccagcagcctgttcctccctatgggatgtatccacccccaggaggaaacccaccctccaggatgccctcatatccgccatacccaggggcccctgtgccgggccagcccatgccaccccccggacagcagcccccaggggcctaccctgggcagccaccagtgacctaccctggtcagcctccagtgccactccctgggcagcagcagccagtgccgagctacccaggatacccggggtctgggactgtcacccccgctgtgcccccaacccagtttggaagccgaggcaccatcactgatgctcccggctttgaccccctgcgagatgccgaggtcctgcggaaggccatgaaaggcttcgggacggatgagcaggccatcattgactgcctggggagtcgctccaacaagcagcggcagcagatcctactttccttcaagacggcttacggcaaggatttgatcaaagatctgaaatctgaactgtcaggaaactttgagaagacaatcttggctctgatgaagaccccagtcctctttgacatttatgagataaaggaagccatcaagggggttggcactgatgaagcctgcctgattgagatcctcgcttcccgcagcaatgagcacatccgagaattaaacagagcctacaaagcagaattcaaaaagaccctggaagaggccattcgaagcgacacatcagggcacttccagcggctcctcatctctctctctcagggaaaccgtgatgaaagcacaaacgtggacatgtcactcgcccagagagatgcccaggagctgtatgcggccggggagaaccgcctgggaacagacgagtccaagttcaatgcggttctgtgctcccggagccgggcccacctggtagcagttttcaatgagtaccagagaatgacaggccgggacattgagaagagcatctgccgggagatgtccggggacctggaggagggcatgctggccgtggtgaaatgtctcaagaataccccagccttctttgcggagaggctcaacaaggccatgaggggggcaggaacaaaggaccggaccctgattcgcatcatggtgtctcgcagcgagaccgacctcctggacatcagatcagagtataagcggatgtacggcaagtcgctgtaccacgacatctcgggagatacttcaggggattaccggaagattctgctgaagatctgtggtggcaatgactgaacagtgactggtggctcacttctgcccacctgccggcaacaccagtgccaggaaaaggccaaaagaatgtctgtttctaacaaatccacaaatagccccgagattcaccgtcctagagcttaggcctgtcttccacccctcctgacccgtatagtgtgccacaggacctgggtcggtctagaactctctcaggatgccttttctaccccatccctcacagcctcttgctgctaaaatagatgtttcatttttctgactcatgcaatcattcccctttgcctgtggctaagacttggcttcatttcgtcatgtaattgtatatttttatttggaggcatattttcttttcttacagtcattgccagacagaggcatacaagtctgtttgctgcatacacatttctggtgagggcgactgggtgggtgaagcaccgtgtcctcgctgaggagagaaagggaggcgtgcctgagaaggtagcctgtgcatctggtgagtgtgtcacgagctttgttactgccaaactcactcctttttagaaaaaacaaaaaaaaagggccagaaagtcattccttccatcttccttgcagaaaccacgagaacaaagccagttccctgtcagtgacagggcttcttgtaatttgtggtatgtgccttaaacctgaatgtctgtagccaaaacttgtttccacattaagagtcagccagctctggaatggtctggaaatgtca

SEQ ID NO: 2 - protein, 505 aa, Homo sapiens
MSYPGYPPPPGGYPPAAPGGGPWGGAAYPPPPSMPPIGLDNVATYAGQFNQDYLSGMAANMSGTFGGANMPNLYPGAPGAGYPPVPPGGFGQPPSAQQPVPPYGMYPPPGGNPPSRMPSYPPYPGAPVPGQPMPPPGQQPPGAYPGQPPVTYPGQPPVPLPGQQQPVPSYPGYPGSGTVTPAVPPTQFGSRGTITDAPGFDPLRDAEVLRKAMKGFGTDEQAIIDCLGSRSNKQRQQILLSFKTAYGKDLIKDLKSELSGNFEKTILALMKTPVLFDIYEIKEAIKGVGTDEACLIEILASRSNEHIRELNRAYKAEFKKTLEEAIRSDTSGHFQRLLISLSQGNRDESTNVDMSLAQRDAQELYAAGENRLGTDESKFNAVLCSRSRAHLVAVFNEYQRMTGRDIEKSICREMSGDLEEGMLAVVKCLKNTPAFFAERLNKAMRGAGTKDRTLIRIMVSRSETDLLDIRSEYKRMYGKSLYHDISGDTSGDYRKILLKICGGND